(19) United States
(12) Patent Application Publication
(10) Pub. No.: US 2011/0118557 A1
(43) Pub. Date: May 19, 2011

(54) Intelligent User Interface For Medical Monitors

(75) Inventors: Edward M. McKenna, Boulder, CO (US); Clark R. Baker, JR., Newman, CA (US)

(73) Assignee: Nellcor Purifan Bennett LLC, Boulder, CO (US)

(21) Appl. No.: 12/949,269

(22) Filed: Nov. 18, 2010

Related U.S. Application Data
(60) Provisional application No. 61/262,445, filed on Nov. 18, 2009.

(51) Int. Cl. A61B 5/00

(52) U.S. Cl. 600/300

(57) ABSTRACT

An intelligent learning process for a user interface of a medical monitor is disclosed. The medical monitor may record user statistics and cluster groups based on settings, configurations, and actions captured by the user statistics. The medical monitor may create classes of users based on the groups and then classify users into classes based on the user statistics. The user interface of the monitor may be adapted based on the user's class. In other embodiments, a central station may access user statistics from multiple monitors and adapt a user interface for the monitors based on the statistics.

### RELATED APPLICATION

This application claims the benefit of U.S. Provisional Application No. 61/262,445, filed Nov. 18, 2009, which application is hereby incorporated by reference.

### BACKGROUND

The present disclosure relates generally to medical monitoring systems and, more particularly, to configuration and operation of medical monitors.

This section is intended to introduce the reader to aspects of the art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. A medical monitoring system may include a monitor that receives signals from various types of optical, electrical, and acoustic sensors. These monitors may display various physiological parameters to a caregiver via a display. However, the monitors may not consistently display the desired physiological parameters, requiring the caregiver to navigate the monitor's user interface to find the physiological parameters of interest. Further, some caregivers may be more proficient at using the user interface of a monitor than other caregivers. Finally, the monitor may not by easily configurable for different care environments or users.

### BRIEF DESCRIPTION OF THE DRAWINGS

Advantages of the disclosure may become apparent upon reading the following detailed description and upon reference to the drawings in which:

FIG. 1 depicts a medical monitoring system in accordance with an embodiment of the present disclosure;

FIG. 2 is a block diagram of the multi-parameter monitor of FIG. 1 in accordance with an embodiment of the present disclosure;

FIG. 3 is a block diagram of the display screens of a user interface of a multi-parameter monitor in accordance with an embodiment of the present disclosure;

FIG. 4 is a block diagram depicting an intelligent learning process of a multi-parameter monitor in accordance with an embodiment of the present disclosure;

FIG. 5 is a block diagram depicting an intelligent learning process of a multi-parameter monitor in accordance with another embodiment of the present disclosure;

FIG. 6 depicts a system having a central station and multiple monitors in accordance with an embodiment of the present disclosure; and

FIG. 7 is a block diagram of an intelligent learning process of the central station of FIG. 6 in accordance with an embodiment of the present disclosure.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

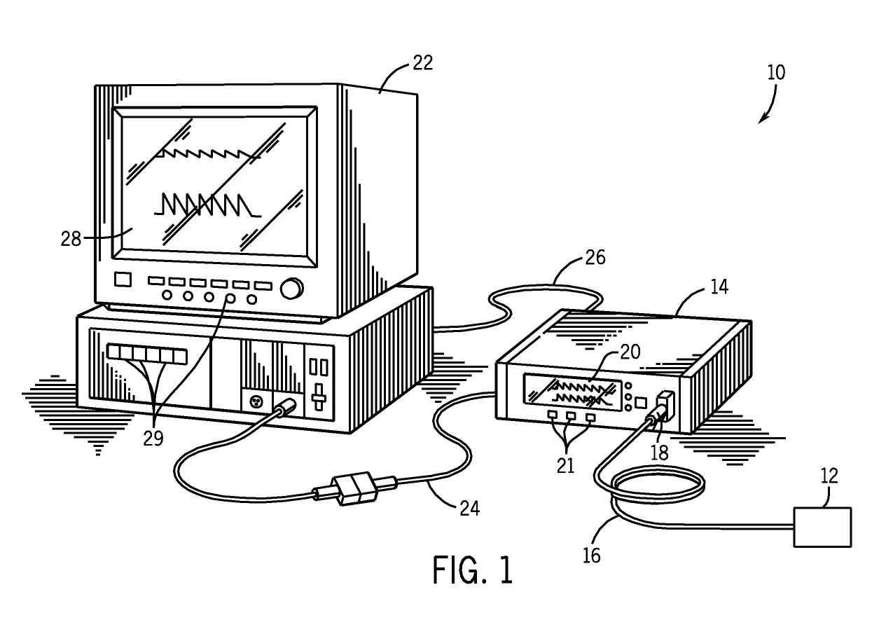

FIG. 1 depicts a medical monitoring system 10 having a sensor 12 coupled to a monitor 14 in accordance with an embodiment of the present disclosure. The sensor 12 may be coupled to the monitor 14 via sensor cable 16 and sensor connector 18, or the sensor 12 may be coupled to a transmission device (not shown) to facilitate wireless transmission between the sensor 12 and the monitor 14. The monitor 14 may be any suitable monitor, such as those available from Nellcor Puritan Bennett, LLC. The monitor 14 may be configured to calculate physiological parameters from signals received from the sensor 12 when the sensor 12 is placed on a patient. In some embodiments, the monitor 14 may be primarily configured to determine, for example, blood and/or tissue oxygenation and perfusion, pulse rate, respiratory rate, respiratory effort, continuous non-invasive blood pressure, cardiovascular effort, glucose levels, level of consciousness, total hematocrit, and/or hydration. Further, the monitor 14 includes a display 20 configured to display information regarding the physiological characteristics, information about the system, and/or alarm indications.

The monitor 14 may include various input components 21, such as knobs, switches, keys and keypads, buttons, touchpad, touch screen, microphone, camera, etc., to provide for operation and configuration of the monitor. As explained further below, such input components 21 may allow a user to navigate a user interface of the monitor 14, configure the monitor 14, and select/deselect information of interest.

Furthermore, to upgrade conventional operation provided by the monitor 14 to provide additional functions, the monitor 14 may be coupled to a multi-parameter patient monitor 22 via a cable 24 connected to a sensor input port or via a cable 26 connected to a digital communication port. In addition to the monitor 14, or alternatively, the multi-parameter patient monitor 22 may be configured to calculate physiological parameters and to provide a central display 28 for information from the monitor 14 and from other medical monitoring devices or systems. For example, the multi-parameter patient monitor 22 may be configured to display a patient's blood pressure on the display 28. The monitor may include various input components 29, such as knobs, switches, keys and keypads, buttons, touchpad, touch screen, microphone, camera, etc., to provide for operation and configuration of the monitor 22. As explained further below, such input components 29 may allow a user to navigate a user interface of the monitor 22, configure the monitor 22, and select/deselect information of interest. In some embodiments, the display 28 may be a touchscreen having software input components 29, such that a user may operate and configure the monitor 22 via the display 28. In addition, the monitor 14 and/or the multi-parameter patient monitor 22 may be connected to a network to enable the sharing of information with servers or other workstations.

The sensor 12 may be any sensor suitable for detection of any physiological characteristic. The sensor 12 may include optical components (e.g., one or more emitters and detectors), acoustic transducer or microphone, electrode for measuring electrical activity or potentials (such as for electrocardiography), pressure sensors, motion sensors, temperature sensors, etc. The sensor 12 may be a bandage-style sensor having a generally flexible sensor body 12 to enable conformable application of the sensor 10 to a sensor site on a patient. The sensor 12 may be secured to a patient via adhesive on the underside of the sensor body 12 or by an external device such as headband or other elastic tension device. In other embodiments, the sensor 12 may be a clip-type sensor suitable for application on an appendage of a patient, e.g., a digit, an ear, etc. In yet other embodiments, the sensor 12 may be a configurable sensor capable of being configured or modified for application to different sites.

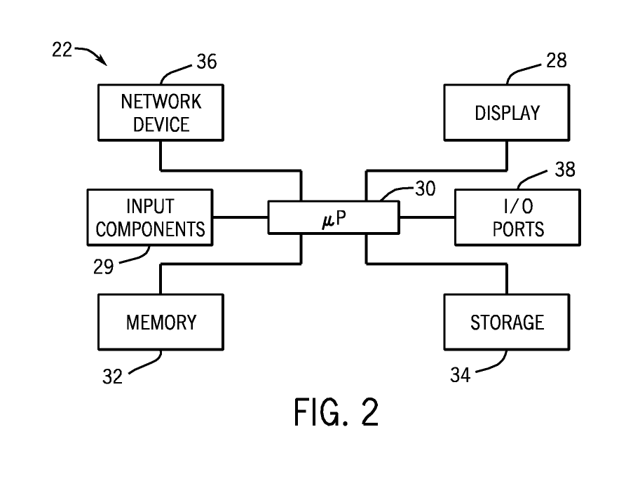

FIG. 2 is a block diagram of the multi-parameter patient monitor 22 in accordance with an embodiment of the present disclosure. As mentioned above, the monitor 22 includes a display 28 and input components 29. Additional components of the monitor 22 illustrated in FIG. 2 are a microprocessor 30, memory 32, storage 34, network device 36, and I/O ports 38. As mentioned above, the user interface may be displayed on the display 28, and may provide a means for a user to interact with the monitor 22. The user interface may be a textual user interface, a graphical user interface (GUI), or any combination thereof, and may include various screens and configurations. The processor(s) 30 may provide the processing capability required to execute the operating system, monitoring algorithms for determining physiological parameters, the user interface, and any other functions of the monitor 22.

The monitor 22 may also include a memory 32. The memory 32 may include a volatile memory, such as RAM, and a non-volatile memory, such as ROM. The memory 32 may store a variety of information and may be used for a variety of purposes. For example, the memory 32 may store the firmware for the monitor 22 and/or any other programs or executable code necessary for the monitor 22 to function. In addition, the monitor 22 may be used for storing data during operation of the monitor 22.

The monitor 22 may also include non-volatile storage (not shown), such as ROM, flash memory, a hard drive, any other suitable optical, magnetic, or solid-state storage medium, or a combination thereof. The non-volatile storage may store data such as software, patient information, user information, user statistics (as discussed further below) and any other suitable data.

The monitor 22 depicted in FIG. 2 also includes a network device 36, such as a network controller or a network interface card (NIC). In one embodiment, the network device 36 may be a wireless network device providing wireless connectivity over any 802.11 standard or any other suitable wireless networking standard. The monitor may also include input/output ports 38 to enable communication with external devices, such as the patient monitor 14 and/or the sensor 12. The input/output ports 38 may include the sensor input port for connection of the cable 24 and a digital communication port for connection of the cable 26.

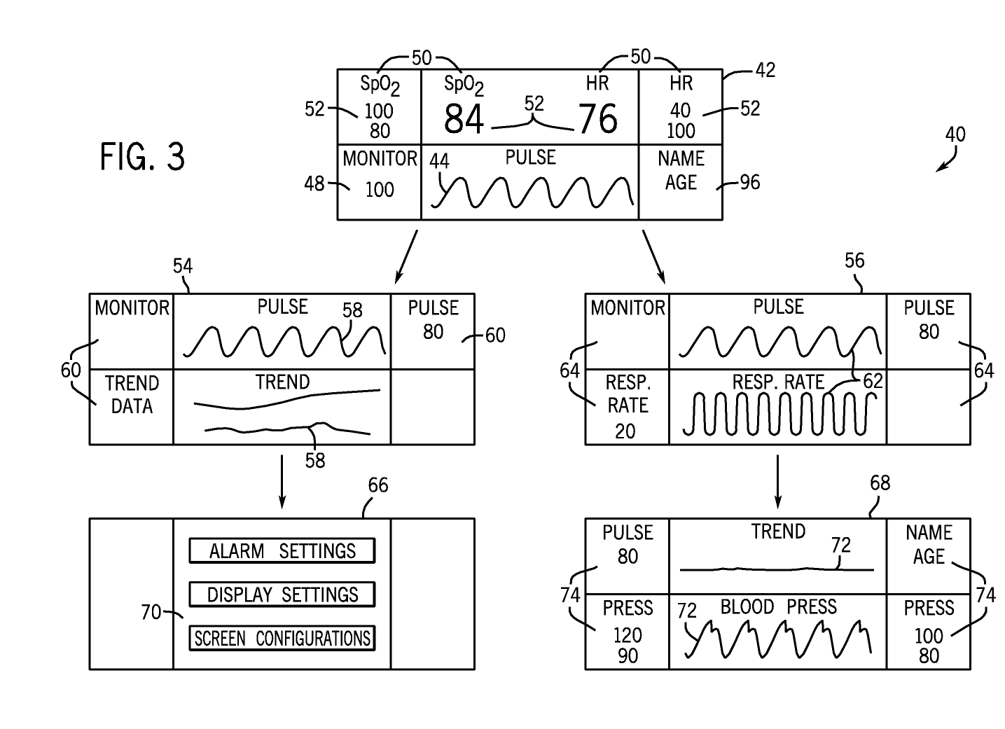

As mentioned above, the multi-parameter monitor 22 may include a user interface to enable a user of the monitor 22 to monitor and control the sensor 12 and monitor any physiological parameters or other information accessible via the monitor 22. FIG. 3 depicts a block diagram of screens 40 of a user interface of the multi-parameter patient monitor 22 in accordance with an embodiment of the present disclosure. The monitor 22 may include a first screen 42 displayed on the display 28. The first screen 42 may be the default screen displayed when the monitor 22 is in normal operation, such as receiving signals from the sensor 12 and displaying sensor information and patient information. It should be appreciated that access to the first screen 42 and the user interface of the monitor 22 may be restricted through any suitable technique, such as requiring users to enter login information, identification of users via an identification device, such as a barcode, RFID tag, or other identification device.

The first screen 42 may display various plethysmographic waveforms 44 correlating to various physiological parameters, such as blood oxygen saturation, EKG, etc. The first screen 42 may also display patient information 46, e.g., the patient's name, age, condition, caregiver, or any other suitable information. Further, the first screen 42 may also display other information 48, such as care environment information, monitor information (e.g., type, version, etc.) and caregiver information. The first screen 42 of the monitor 22 may also provide any other text information 50 and/or numeric information 52 relating to the monitor, sensor, patient, and physiological parameters, such as identification of a physiological parameters and the corresponding numeric value of that parameter.

In order to operate and configure the monitor 22, a caregiver may desire to view additional information regarding the monitor 22, sensor 12, physiological parameters, and/or patient. Additionally, the caregiver may desire to add or remove user interface elements to the first screen 42. The caregiver may access screens 54 and 56 by interaction with the input components 29. For example, to access the screen 54, the user may execute one or more keystrokes, (e.g., one key, sequence of keys, or combination of keys) on the monitor 22. Similarly, to access the screen 56, the caregiver may execute a second one or more keystrokes.

Each of the screens 54 and 56 may display information, such as additional physiological parameters, additional patient information, additional sensor information, etc., monitored by the monitor 22. For example, the screen 54 may include graphical data 58 and text and/or numeric data 60. The screen 56 may also include graphical data 62 and text or numeric data 64. A caregiver may desire to move some or all of the data displayed on the screens 54 and 56 to the first screen 42. Thus, a user may alter a setting in the user interface to select, for example, text or numeric data 60 and configure the monitor such that this text and/or numeric data 60 is displayed on the first screen 42.

A user of the monitor 22 may access screens 66 and 68, again through selection of various input components 29. To access screen 66, for example, a user may execute additional keystrokes so that the screen 66 is then displayed on the display 28 of the monitor 22. To access screen 68, a caregiver may execute different keystrokes so that the screen 68 is displayed on the display 28 of the monitor 22.

Each screen 66 and 68 may display information viewable by the user. In other embodiments, the screens 66 and 68 may provide access to settings or configurations to allow enable configuration of the monitor 22. For example, the screen 66 may include settings 70 to allow configuration of the monitor 22, so that the user may select, deselect, or adjust various settings and/or configurations of the monitor 22. The screen 68 may include graphical information 72 and text and/or numeric data 74. Thus, by accessing screens 54, 56, 66, and 68 through selection of input components 29 (user “actions”), a user may “drilldown” into the user interface to view information or access settings or configurations of the monitor 22. Collectively, these settings, configurations, and actions accessed and executed by the user may be referred to as user statistics.

It should be appreciated that FIG. 3 is merely representative of a user interface of the monitor 22. In other embodiments, any number of screens and arrangements may be accessible to a user, and screens may display any type of information and/or allow access to any settings or configurations.

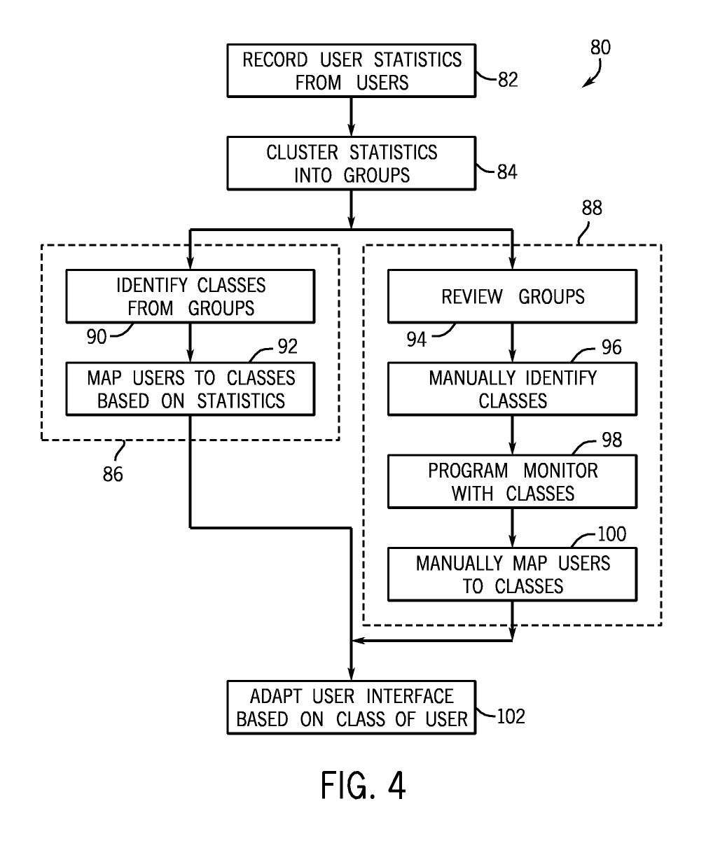

FIG. 4 is a block diagram depicting an intelligent learning process 80 of the monitor 22 in accordance with an embodiment of the present disclosure. As described in detail below, the intelligent learning process of the monitor 22 may adapt the user interface of the monitor 22, such as the screens displayed on the monitor 22 and the information displayed on such screens, by identifying particular users and/or classes of users based on user statistics of the monitor 22. Any or all steps of the process 80 may be implemented in code stored on a tangible machine-readable medium, such as the storage 34 of the monitor 22.

Initially, the user's statistics (e.g. a user's selections of settings, configurations, and a user's actions) on the monitor 22 may be recorded to build a database (or other suitable data structure) of user statistics (block 82). Any type of user statistic may be recorded. Such statistics may include, but are not limited to: information accessed by the user, settings and configurations selected by the user, configuration of various screens (such as addition or removal of physiological parameters to be displayed), alarm settings, alarm reductions, etc. Any interaction between a user and the monitor 22 may be recorded by the monitor 22 and recorded as user statistics.

After recording user statistics, the monitor 22 may cluster the user statistics into different groups (block 84). These groups may be based on actions, settings, and/or configurations of the monitor 22 that are commonly used together, as captured by the recorded user statistics. For example, if a certain physiological parameter is commonly added for display in the first screen of the user interface, this setting may be clustered into a first group in combination with other actions, settings, or combinations that are commonly used with this display of the physiological characteristic. In another example, if certain keystrokes are commonly used with a certain configuration, such as to access other screens, these keystrokes may be clustered into a group with the configurations.

Any number of groups may be formed that include any number of settings, actions, and/or configurations based on the user statistics. Additionally, groups may include overlapping settings, actions, and/or configurations. The number of groups and the specificity of the clustering may be set at a default value on the monitor 22 and may be modified by a user via a setting on the monitor 22.

After clustering the user statistics into groups, the monitor may create user classes based on the groups and classify users into different classes based on each user's statistics. The classification may be automatically performed by the monitor 22 (referred to as unsupervised path 86) or manually performed by a user (referred to as supervised path 88). The selection of the unsupervised path 86 or supervised path 88 may be selected on the monitor 22 by a user, one selection may be a default, or only one selection may be present on a particular monitor.

In the unsupervised path 86, the monitor 22 automatically classifies users. Initially, the monitor may create one or more classes based on the groups of user statistics (block 90). Each class may be based on one or more groups of user statistics, or each class may be based on one group or a portion of a group. The classes may be selected to encompass commonly used actions, settings, and configurations of the monitor 22.

After identifying the classes, the monitor 22 may assign users into the identified classes based on each user's statistics (block 92). Each class may include one or more users, and in some embodiments users may be assigned to multiple classes. For example, if a first class contains two groups A and B, and a user's statistics primarily fall into a group A, that user may be classified into the first class. If a second class contains group C, and a user's statistics primarily fall into group C, that user may be assigned to the second class.

In the supervised path 88, a user may manually create the classes on the monitor 22. Initially, a user can review the groups (i.e., review the results of the clustering) and review which user statistics are clustered into which groups (block 94). If desired, the user can manually adjust the clustering by adding or removing settings, actions, and/or configuration to and from groups. After reviewing the groups, a user may manually identify and create classes based on the groups (block 96). The user may identify and create the classes on the monitor and assign groups to each class (block 98). As mentioned above, each class may be based on one or more groups of user statistics, or each class may be based on one group or a portion of a group. Finally, users may be manually assigned to the created classes (block 100). Again, as noted above, each class may include one or more users, and in some embodiments users may be assigned to multiple classes.

After completion of the supervised path 88 or unsupervised path 86, the monitor 22 may automatically provide the settings, actions, and configurations for each user according to the user's classification. For example, after a user logs into the monitor 22, the monitor 22 may determine the user's class and adjust the user interface based on the settings specific to the class. The monitor 22 may also provide any configurations based on the user's class. For example, if the class indicates that certain physiological parameters should be displayed on the first screen of the monitor 22, the monitor 22 may automatically display those characteristics after the user logs in, so that the user does not need to reconfigure the monitor 22. Additionally, further settings related to the display of the physiological parameter, such as units, granularity, refresh rate, etc. may be automatically set based on the user's class.

Additionally, the monitor 22 may reconfigure various actions based on the user's class. The monitor 22 may reconfigure the input components 29 and/or the user interface to lower the acuity of the monitor (e.g., by reducing the keystrokes used to access various screens or settings). For example, as noted above, in some embodiments the user interface of the monitor 22 may include any number of nested screens accessible by one or more keystrokes. In such an example, the class may indicate that users of that class commonly access the screen 68. The monitor 22 may reconfigure the keystrokes (or other action) required to access the screen 68, so that instead of a sequence of four keystrokes, for example, the screen 68 may be accessed via a sequence of two keystrokes. The monitor 22 may reconfigure any such keystrokes to provide easier access to various screens and/or settings for a class. In some embodiments, the monitors may store class statistics, by further recording various actions, settings, configurations, etc. used by a user's of a certain class.

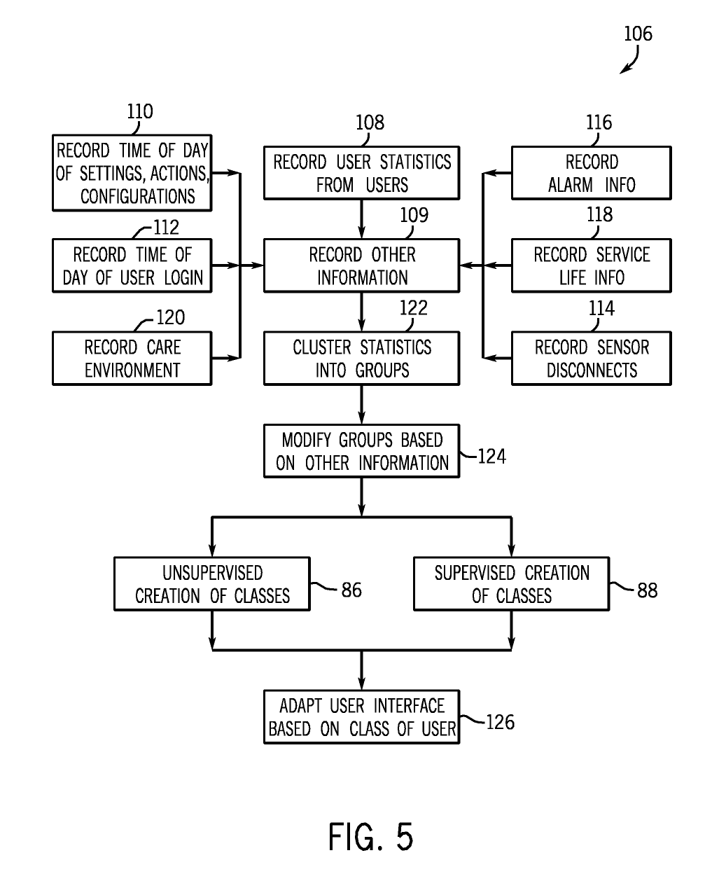

In other embodiments, the monitor 22 may incorporate other types of information into the determination of groups and/or classes. This information may be programmed into the monitor by a user, determined from various monitor settings, or determined from user statistics. FIG. 5 is a block diagram depicting operation of an intelligent learning process 106 of the monitor 28 in accordance with another embodiment of the present disclosure. During operation, as discussed above, statistics for users of the monitor 22 may be recorded and stored in a database (or other data structure), such as on the storage 34 (block 108).

In addition, as shown in FIG. 5, the monitor 22 may record alternative or additional information (block 109). These statistics may include the time of day that various settings, actions, and configurations are taken (block 110) or the time of day that various users login to the monitor 22 (block 112). The monitor 22 may record the number of times a sensor coupled to the monitor 22 is disconnected and connected to the monitor 22 for a given user (block 114). The monitor 22 may record the number and severity of alarms during a period of time (block 116). Additionally, the monitor 22 may record the overall service life-time of the monitor 22, and may record how long the monitor 22 has monitored each patient and/or the current patient (block 118).

Further in some embodiments, the monitor 22 may record the type of care environment where the monitor is in use (block 120), e.g., Intensive Care Unit (ICU), general care, operating room etc. In one embodiment, the type of care environment may be manually entered into the monitor 22 by a user. In other embodiments, the monitor 22 may automatically determine the type of care environment based on the user statistics and/or the alarms or other data relating to the physiological parameters being monitored. For example, an ICU care environment may use more sensitive alarms, and may include more displayed physiological parameters, such as a patient's respiratory rate.

After collection of these user statistics and other information, the monitor 22 may proceed to cluster groups of commonly used settings, configurations, and actions based on the user statistics (block 122), such as described above in block 84 of FIG. 4. The data recorded by the monitor may also be used in selecting various settings, actions, and configurations (block 124). For example, when grouping certain settings and configurations, the monitor may select or deselect certain settings or configurations based on the type of care environment. For example, if the type of care environment is an operating room, certain groups may include settings that smooth out the plethysmographic waveforms displayed on the monitor 22. After clustering groups, the monitor 22 may proceed to create classes and classify users according to the supervised path 88 or unsupervised path 86 described above in FIG. 4. Theses classes may incorporate the additional settings, configurations, and actions clustered to each group that may be based on the additional information.

After completion of the supervised path 88 or unsupervised path 86, the monitor 22 may adapt the user interface by automatically enabling the settings, actions, and configurations for each user according to the user's classification (block 126). Again, based on the additional information used by the monitor 22, the classes may include additional settings, actions, and configurations based on such additional information. For example, if the monitor 22 records a specific care environment, certain settings may be selected based on the care environment to adapt the user interface to the care environment. In another example, if certain settings and configurations are commonly selected during specific period of time during the day, the user interface may be adapted based on the selected settings and configurations during that period of time. Additionally, as also discussed above, the monitor 22 may reconfigure various actions based on the user's class. The monitor 22 may reconfigure the input components 29 and/or the user interface to lower the acuity of the monitor (e.g., by reducing the keystrokes used to access various screens or settings). This reconfiguration may also be based on the additional information stored by the monitor 22.

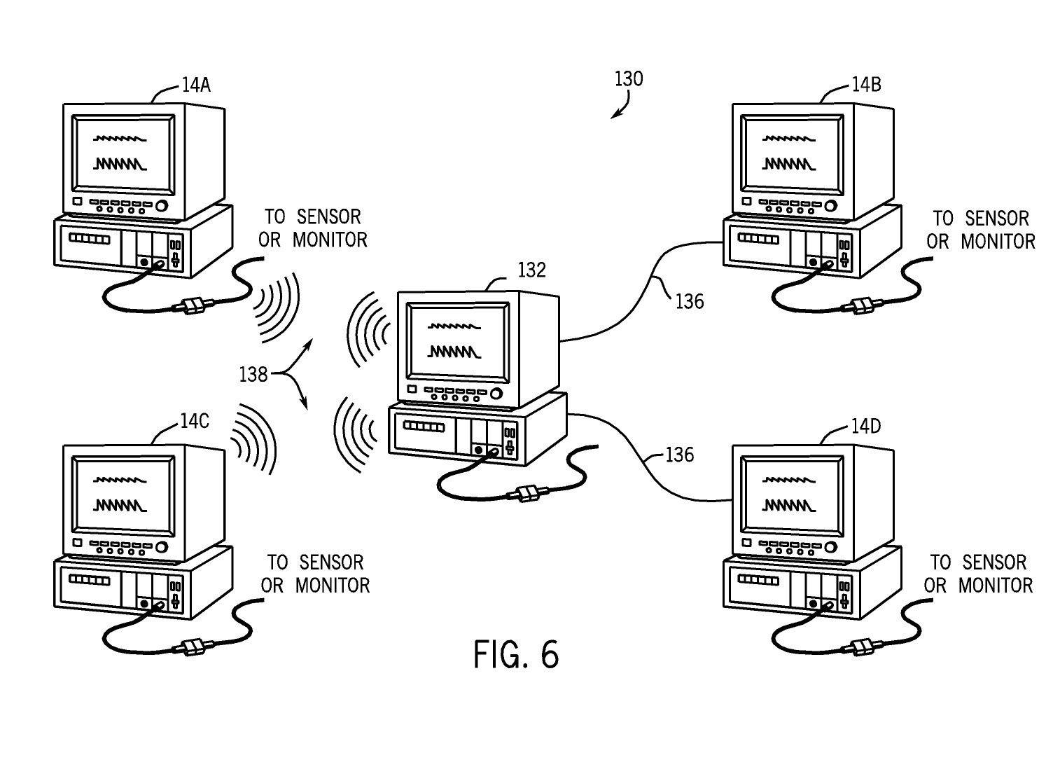

In other embodiments, a central station may record, analyze, and adapt the user interface across multiple monitors. FIG. 6 depicts a system 130 having a central station 132 in communication with multiple monitors 14A, 14B, 14C, and 14D in accordance with another embodiment of the present disclosure. The central station 130 may any suitable electronic device, such as a monitor, computer etc., and may include any or all of the components illustrated above in FIG. 2, such as a processor, memory, and non-volatile storage. In one embodiment, the central station 132 may be an Oxinet® central station available from Nellcor Puritan Bennett LLC. The central station 132 may be coupled to some of the monitors 14B and 14D via physical network connections 136, such as an Ethernet network or any other suitable network. The central station 132 may also be coupled to some of the monitors 14A and 14C via wireless connections 138, such as wireless Ethernet or other suitable wireless network.

The central station 132 may provide a user interface or updates to a user interface for the monitors 14A, 14B, 14C, and 14D. A user interface may be created and/or configured on the central station 132 and sent to all of the monitors 14A, 14B, 14C, and 14D so that each monitor provides an identical user interface. For example, the user interface on the central station 132 may be configured to display certain screens, certain information on such screens, and/or the action of keystrokes for navigation in the user interface.

Each monitor 14A, 14B, 14C, and 14D may be coupled to one or more monitors or sensors, such as in the system illustrated above in FIG. 1. The monitors 14A, 14B, 14C, and 14D may send information such as patient data, physiological parameter data, and any other data to the central station 132. Additionally, the monitors 14A, 14B, 14C, and 14D may send user statistics, such as settings, actions, and configurations to the central station 132. The central station 132 may record these user statistics in a database (or other suitable data structure) stored on the central station 132. Additionally, or alternatively, the monitors 14A, 14B, 14C, and 14D may store the user statistics. These stored user statistics may be accessed by the central station 132 over the network connections 136 and/or 138.

The central station 132 may adapt a user interface based on the user statistics and provide the monitors 14A, 14B, 14C, and 14D with the adapted user interface. The central station 132 may provide a single adapted user interface configuration to each monitor 14A, 14B, 14C, and 14D, or the central station 132 may selectively send different adapted user interface configurations to different monitors or groups of monitors 14A, 14B, 14C, and 14D. Additionally, or alternatively, the central station 132 may send a user interface adapted to a specific user to any of the monitors 14A, 14B, 14C, and 14D that are currently being or will be accessed by that user, thus providing an adapted user interface for each user of any one of the monitors 14A, 14B, 14C, and 14D.

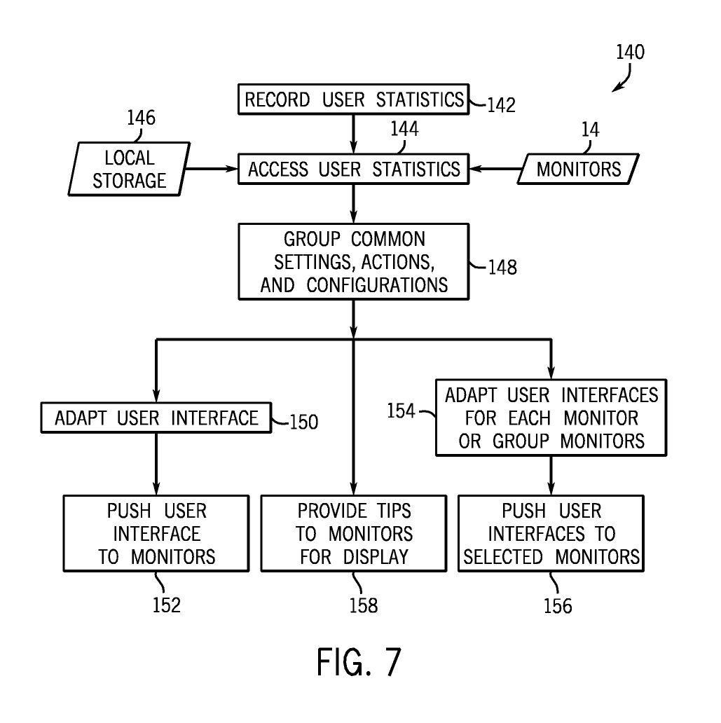

FIG. 7 is a block diagram depicting an intelligent learning process 140 of the central station 132 and system 130 of FIG. 6 in accordance with another embodiment of the present disclosure. During normal operation of the system 130, the user statistics may be recorded by each of the monitors 14A, 14B, 14C, and 14D of the system. Such statistics may be recorded in a database (or other suitable data structure) of user statistics and stored centrally on the central station 132 or on each of the monitors 14A, 14B, 14C, and 14D, as described above. Any type of user statistics may be recorded. Such statistics may include, but are not limited to: information accessed by the user, configuration parameters selected by the user, configuration of various screens (such as addition or removal of physiological characteristic displays to and from screens), monitor settings selected by the user, actions (such as keystrokes) taken by the user, etc. Any interaction between a user and the monitors may be recorded by each monitor as a user statistic.

After the collection of user statistics, the central station 132 may retrieve the user statistics for further processing (block 144). In one embodiment, the central station 132 may store the user statistics from each monitor locally, such as in a non-volatile storage and may access the user statistics from local storage (block 146). In other embodiments, the user statistics for each monitor 14A, 14B, 14C, and 14D may be stored on the each of the monitors, and the central station 132 may access the user statistics on each monitor 14A, 14B, 14C, and 14D.

After accessing the user statistics, the central station may cluster commonly used settings, action, and configurations into various groups (block 148), as described above in FIGS. 5 and 6. These groups may be based on statistics for one user or multiple users. For example, if one user of the monitors 14A appears to provide detailed customization of the user interface, the central station 132 may cluster those settings, actions, and configurations captured in those user statistics into a group. Thus, a user who is proficient in customizing the user interface provided in the system 130 enables the central station 132 to select a group that captures that proficiency of that user. As discussed below, that proficiency may be used to adapt the user interfaces of all the monitors 14A, 14B, 14C, and 14D in the system 130.

After grouping the setting, actions, and configurations, the central station 132 may adapt a common user interface for the monitors 14A, 14B, 14C, and 14D (block 150). As discussed above, this adaptation may include modifying the user interface based on the settings, actions, and configurations of a group. For example, if specific settings indicate that certain physiological parameters are commonly displayed in a certain format, the central station 132 may customize the user interface so that the user interface automatically displays physiological parameters in the format by default. If certain configurations, such as units, alarm settings, etc. are also clustered together with certain settings of a group, the central station 132 may apply those settings to the customized user interface. In another example, as also mentioned above, the central station 132 may reconfigure the keystrokes used to access certain screens, settings, or other elements of the user interface. After adapting the user interface, the central station 132 may “push” the user interface to each of the monitors 14A, 14B, 14C, and 14D over the network (block 152), so that each monitor 14A, 14B, 14C, and 14D is updated with the new user interface. If any of the monitors 14A, 14B, 14C, and 14D are currently in use, such a monitor may receive the user interface but delay installation until the monitor is not in use. In other embodiments, the monitors 14A, 14B, 14C, and 14D may “pull” the adapted user interface from the central station, such as by periodically checking the central station 132 for an updated version of the user interface.

In some embodiments, the central station 132 may adapt a different user interface for each monitor or group of monitors (block 154). For example, the statistics received from a group of monitors may indicate common usage, common users, or other common factors that suggest the use of an adapted user interface for this group of monitors and not for the remaining monitors. In such an embodiment, the central station 132 may “push” an adapted user interface to the selected monitor or group of monitors (block 156). Other adapted user interfaces may be pushed to other monitors or groups of monitors, again based on common usage, users, etc. In such embodiments, the monitors 132 may instead “pull” the adapted user interface from the central station 132 by periodically checking for updates. The central station 132 may earmark an adapted user interface for a specific monitors or group of monitors by associating a unique identifier for each monitor with the adapted user interface intended for use by such monitors.

In some embodiments, the central station 132 may provide instructional text (i.e., “tips”) for display on one or more of the monitors 132. This instructional text 158 may be based on the grouping of settings, actions, and configurations performed by the central station 132. For example, if a particular setting is commonly used by the majority of users, instructional text may be provided to each monitor 14A, 14B, 14C, and 14D that suggests use of that setting. In another example, the instructional text may also suggest additional or reconfigured keystrokes for accessing settings and/or configurations, such as when keystrokes are reconfigured for an adapted user interface. The monitors 14A, 14B, 14C, and 14D may be configured to display such instructional text at startup, at user login, periodically, or at any other event and/or interval.

What is claimed is:

1. A system, comprising:
a medical monitor coupled to a sensor, wherein the medical monitor is configured to display one or more physiological parameters, store user statistics of one or more users, and adapt a user interface of the monitor based on the user statistics.

2. The system of claim 1, wherein the monitor is configured to cluster commonly used settings, configurations, and/or actions of the user statistics into one or more groups.

3. The system of claim 1, wherein the monitor is configured to determine a care environment for the monitor based on the statistics.

4. The system of claim 3, wherein the monitor is configured to adapt a user interface of the monitor based on the statistics and the care environment.

5. The system of claim 1, wherein adapting the user interface comprises modifying the information displayed on a first screen of the user interface.

6. The system of claim 1, wherein adapting the user interface comprises reconfiguring the keystrokes used to access one or more elements of the user interface.

7. The system of claim 1, wherein adapting the user interface comprises modifying one or more alarms.

8. The system of claim 1, comprising creating a plurality of classes based on the user statistics.

9. The system of claim 8, comprising wherein the monitor is configured to classify the one or more users into one or more of the plurality of classes based on the user statistics.

10. The system of claim 9, wherein the monitor is configured to adapt the user interface for the one or more users based on the class of the one or more users.

11. A system, comprising:
a central station; and
a plurality of medical monitors coupled to the central station, and each comprising a user interface,
wherein the central station is configured to access user statistics from at least one of the plurality of medical monitors and adapt the user interface of one or more of the plurality of medical monitors based on the user statistics.

12. The system of claim 11, wherein central station is configured to store the user statistics.

13. The system of claim 11, wherein each of the plurality of medical monitors is configured to store the user statistics.

14. The system of claim 11, wherein the central station is configured to push the user interface to one or more of the plurality of medical monitors.

15. The system of claim 11, wherein the central station is configured to provide instructional text to the plurality of medical monitors for display on one or more of the plurality of medical monitors.

16. The system of claim 11, wherein the central station is configured to cluster commonly used settings, configurations, and/or actions of the user statistics into one or more groups.

17. A method, comprising:
storing a plurality of user statistics on a medical monitor;
determining a plurality of classes based on the user statistics; and
adapting a user interface of the monitor based on the classes.

18. The method of claim 17, comprising classifying users into one or more of the plurality of classes based on the user statistics.

19. The method of claim 18, comprising storing at least one of the care environment of the monitor, the service life of the monitor, the connection of sensors to the monitor and the disconnection of sensors to the monitor on the medical monitor.

20. The method of claim 19, wherein adapting the user interface comprises reconfiguring the keystrokes used to access one or more elements of the user interface.
